(19) United States
(12) Patent Application Publication
(10) Pub. No.: US 2009/0162431 A1
(43) Pub. Date: Jun. 25, 2009

(54) Sustained release formulations containing acetaminophen and tramadol

(76) Inventors: Shuyi Zhang, Parsippany, NJ (US); Jin Wang, Cedar Grove, NJ (US); Shaoling Li, Sunnyvale, CA (US)

Correspondence Address: Joseph A. Fuchs;Rockey, Depke & Lyons, LLC, 233 S. Wacker Drive, Suite 5450, Sears Tower, Chicago, IL, 60606-6306, US

(21) Appl. No.: 12/152,934

(22) Filed: May 19, 2008

Related U.S. Application Data
(63) Continuation-in-part of application No. 10/664,451, filed on Sep. 20, 2003, now Pat. No. 7,374,781.
(60) Provisional application No. 60/412,499, filed on Sep. 21, 2002.

(51) Int. Cl. A61K 9/52; A61K 31/135; A61K 9/14; A61K 9/32

(52) U.S. Cl. 424/457; 514/646; 424/497; 424/468

(57) ABSTRACT

The present invention provides a pharmaceutical dosage form for sustained release of a combination of acetaminophen and tramadol or its salts. The dosage form has an immediate release portion and a sustained release portion. The immediate release portion has about 16%-75% of the drugs. The sustained release portion includes: a. about 25%-84% of the drugs, at least one gelling polymer in an amount by weight of the total formulation of about 6% to 50%. The dosage form releases about 25% to about 60% of the drugs in the first hour, and not less than about 80% of the drugs in the first 24 hours in an intestinal fluid dissolution media using USP dissolution method II with the paddle speed between 50 rpm and 100 rpm.

### CROSS-REFERENCE TO RELATED APPLICATION

This application is a continuation-in-part patent application of U.S. patent application Ser. No. 10/664,451, filed on Sep. 20, 2003, which claims priority to U.S. Provisional Patent Application No. 60/412,499, filed on Sep. 21, 2002, both of these references are incorporated herein by reference and made a part hereof.

### FEDERALLY SPONSORED RESEARCH OR DEVELOPMENT

Not Applicable.

### BACKGROUND OF THE INVENTION

1. Technical Field

The present invention is directed to sustained release dosage forms (formulations) of acetaminophen and tramadol, dissolution and in vivo performance. In particular, the invention provides a drug formulation that delivers a therapeutically effective combination of acetaminophen and tramadol suitable for twice a day or once a day administration and provides at least about 8-12 hours or 16-24 hours of analgesia for pain after the administration of a single dose of the drug formulation. The invention preferably provides a release of analgesically-effective quantities of acetaminophen and tramadol within about 1.5 hour and extended release of analgesically-effective quantities of acetaminophen and tramadol for at least 8-12 hours or 16-24 hours.

2. Background Art

The present invention is directed to a sustained release formulation of acetaminophen and tramadol. Particularly, this invention is related to a formulation and dissolution specifications from a sustained release dosage form of acetaminophen tramadol in connection with an improved dose segment for a better patient compliance.

### BACKGROUND OF THE INVENTION

Acetaminophen with Codeine Phosphate (Tylenol® with Codeine), Hydrocodone Bitartrate (Vicodin®), and Oxycodone (Tylox®) are commonly used analgesic drugs, indicated for the relief of moderate to moderately severe pain. Acetaminophen with codeine or hydrocodone combines the analgesic effects of a centrally acting analgesic, codeine or hydrocodone, with a peripheral analgesic, acetaminophen. Opioids have for many years been used as analgesics to treat severe pain. They, however, produce undesirable side effects and as a result cannot be given repeatedly or at high doses. The side effect problems are well documented in the literature (J. Jaffe and W. Martin in chapter 15, “The Pharmacological Basis of Therapeutics,” editors L. Goodman and A. Gilman, 5th Edition, 245, 1975), which discloses that morphine and its congeners, e.g., codeine, hydrocodone and oxycodone, are opioid agonist analgesics that exhibit side effects such as respiratory depression, constipation, tolerance and abuse liability.

As alternatives to using opioids, non-opioids such as acetaminophen (APAP) and aspirin are used as analgesics. APAP, like aspirin, is not subject to the tolerance, addiction and toxicity of the opioid analgesics. However, APAP and aspirin are only useful in relieving pain of moderate intensity, whereas the opioid analgesics are useful in relieving more intense pain; See Woodbury, D. and Fingl, E. in “The Pharmacological Basis of Therapeutics,” 5th Ed.; Goodman, L. and Gilman, A., Chapter 15, pages 325 (1975).

To reduce the side effect problems of opioids, opioids have been combined with other drugs including non-opioid analgesic agents, which lowers the amount of opioid needed to produce an equivalent degree of analgesia. It has been claimed that some of these combination products also have the advantage of producing a synergistic analgesic effect. For example, A. Takemori, Annals New York Acad. Sci., 281, 262 (1976) discloses that compositions including combinations of opioid analgesics with drugs other than analgesics exhibit a variety of effects, i.e., subadditive (inhibitory), additive or superadditive. R. Taber et al., J. Pharm. Expt. Thera., 169(1), 29 (1969), disclose that the combination of morphine and methadone, another opioid analgesic, exhibits an additive effect. U.S. Pat. No. 4,571,400 discloses that the combination of dihydrocodeine, an opioid analgesic, and ibuprofen, a non-opioid analgesic, provides superadditive effects when the components are within certain ratios. A. Pircio et al., Arch. Int. Pharmacodyn., 235, 116 (1978) report superadditive analgesia with a 1:125 mixture of butorphanol, another opioid analgesic, and APAP, a non-opioid analgesic, whereas a 1:10 mixture did not show any statistically significant superadditive analgesia.

An immediate-release tablet composition comprising a tramadol and acetaminophen, and its use, is disclosed in U.S. Pat. No. 5,336,691. The usual adult dosage is one or two tablets (Ultracet®) every four to six hours. The compositions are pharmacologically useful in treating pain and tussive conditions. The compositions are also subject to less opioid side-effects such as abuse liability, tolerance, constipation and respiratory depression.

The benefits of sustained release dosage forms are well documented. To reduce the dose segment and increase the patient compliance are two of the purposes with sustained release formulations. It has been reported that sustained release oral solid dosage forms of opioid analgesics are provided as multiparticulate systems which are bioavailable and which provide effective blood levels of the opioid analgesic for at least about 24 hours (see U.S. Pat. No. 6,294,195). U.S. Pat. No. 6,126,969 discloses preparing sustained release tablets for acetaminophen using coated acetaminophen particles and uncoated acetaminophen particles. The two particles are compressed into tablets providing a combination of immediate release and sustained release dosage forms.

U.S. Pat. No. 4,850,522 discloses an acetaminophen-sustained release tablet or tablet layer and a method for preparing the same. The '522 discloses forming a wet granulation, using Povidone (PVP) in water or alcohol-water mixture as the granulating fluid. The wet granulation is mixed with acetaminophen, hydroxyethyl cellulose, and a wicking agent e.g., microcrystalline cellulose. This granulation solution is then subjected to drying and milling and is then blended with dry powdered erosion promoter, e.g., pregelatinized starch, a wicking agent, a lubricant (e.g., magnesium stearate and glidant e.g., silicon dioxide), and compressing the resultant granulation tablet. Administration of such tablet results in a slow release of the acetaminophen.

It has previously been known in the art that controlled release compositions of opioid analgesics such as morphine, hydromorphone or salts thereof could be prepared in a suitable matrix. For example, U.S. Pat. No. 4,990,341 describes hydromorphone compositions wherein the dissolution rate in vitro of the dosage form, when measured by the USP Paddle Method at 100 rpm in 900 ml aqueous buffer (pH between 1.6 and 7.2) at 37° C., is between 12.5 and 42.5% (by wt) hydromorphone released after 1 hour, between 25 and 55% (by wt) released after 2 hours, between 45 and 75% (by wt) released after 4 hours and between 55 and 85% (by wt) released after 6 hours.

In the pharmaceutical market today, there are only sustained release dosage forms available for individual analgesic drugs, for example, oxycodone or acetaminophen.

These and other aspects and attributes of the present invention will be discussed with reference to the following drawings and accompanying specification

### BRIEF DESCRIPTION OF THE DRAWINGS

FIG. 1 is a graph plotting mean plasma concentration in (ng/ml) versus time (hr) for acetaminophen for test formulations T1 and T2 and for a reference formulation;

FIG. 2 is a graph plotting mean plasma concentration in (ng/ml) versus time (hr) for tramadol for test formulations T1 and T2 and for a reference formulation; and

FIG. 3 is a graph plotting mean plasma concentration in (ng/ml) versus time (hr) for active M1 for test formulations T1 and T2 and for a reference formulation.

### SUMMARY OF THE INVENTION

### Detailed Description of the Invention

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated.

The present invention contemplates two principal sustained release formulations of a combination of APAP and tramadol. A first sustained release formulation provides for twice-a-day administration, or twelve hour release. A second sustained release formulation provides for once-a-day administration, or a twenty-four hour release.

The present invention provides a method for pain management with these sustained release formulations. For the twelve-hour release formulation, about 25% to about 60% of the total drug is released in the first hour, and not less than about 80% of the total drug is released in the first 12 hours.

For the twenty-four hour release formulation, about 25 to about 60% of the total drug is released in the first 1 hour, and not less than about 80% of the total drug is released in the first 24 hours. The quantity of drug release is determined by placing the formulations in an intestinal fluid using USP dissolution method II with a paddle speed between 50 rpm and 100 rpm.

In a particularly preferred form of the invention, the formulations may be used to provide a depot drug form for controlled release of APAP and tramadol containing pharmaceutical composition. However, the formulations are also useful in connection with a variety of other pharmaceutical or active compositions, including water soluble compositions, water sparingly soluble compositions and water insoluble compositions, and, therefore, the invention should not be considered as being limited by the exact composition and/or nature of the pharmaceutical or other active composition which is released under controlled conditions therefrom.

In a preferred form of the invention, the formulations are prepared in the forms of either tablets or capsules. The controlled release formulations of the invention include a portion for immediate release and a portion for sustained release. In one preferred form of the invention, the immediate release portion will contain from about 16% to about 75% of the total drugs (APAP and tramadol or salts thereof), and the sustained release portion will contain from about 25% to about 84% of total drugs. The sustained release portion will also include at least one gelling polymer. In a preferred form of the invention, the gelling polymer will have a viscosity within the range of from about 60 to about 7,000,000 centipoises, and more preferably from about 100 to about 100,000 centipoises, in a 2% by weight solution at 25° C., as measured by a Brookfield LV viscometer. Suitable gelling polymers include polymeric materials and more preferably homopolymer, copolymers and terpolymers derived from, substituted and unsubstituted, acrylic, methacrylic, methacyrlic acid, methacrylate, ethacrylic acid, and ethacrylate monomers. More specifically, suitable gelling polymers include, for example, hydroxy propyl methylcellulose, hydroxypropyl methylcellulose, hydroxypropyl cellulose, cellulose acetate, hydroxy ethylcellulose, methylcellulose, xantham gums, alginate salts, polyethylene oxide, carboxyvinyl polymer, or a salt of a carboxymethyl cellulose.

The sustained release portion can, optionally, include an enteric coating material selected from homopolymers, copolymers and terpolymers of acrylic and methacrylic acid, cellulose acetate phthalate, cellulose phthalate hydroxy propyl methyl ether, polyvinyl acetate phthalate, hydroxypropyl methylcellulose acetate succinate, cellulose acetate trimellitate, or a shellac.

The active drug contents of APAP and tramadol or salts thereof in the overall tablet formulation may preferably range from about 40% to about 85% by weight. The total amount of the gelling polymers in the overall formulation may preferably range from about 6% to about 50% by weight. The total amount of the enteric polymer in the overall tablet formulation may preferably range from about 5% to about 40% by weight.

The dosage forms in this invention include tablets and capsules containing one form or combination of pellets, granules, powders and 2-12 of mini-tablets.

The following non-limiting Examples are provided to exemplify the invention but not to limit the invention.

### EXAMPLE 1

### Preparation of Sustained Released Dosage Forms Containing Acetaminophen and Tramadol

TABLE 1
Composition (%) of single unit immediate-release and sustained-release
tablets, respectively, containing Acetaminophen and Tramadol

| | | Immediate | Sustained |
|---|---|---|---|
| | Ingredient | Release | Release |
| 1 | Acetaminophen | 200.0 mg | 300 mg |
| 2 | Tramadol Hydrochloride | 20.0 mg | 30.0 mg |
| 3 | Microcrystalline Cellulose | 50.4 mg | 8.8 mg |
| 4 | Povidone K-90 | 19.2 mg | 17.6 mg |
| 5 | Starch | 19.2 mg | — |
| 6 | Croscarmellose Sodium | 4.8 mg | — |
| 7 | Sodium Alginate (Keltone LV) | — | 35.2 mg |
| 8 | Hydroxypropyl Methylcellulose | — | 39.6 mg |
| | (Methocel K4M) | | |
| 9 | Colloidal Silicon Dioxide | 3.2 mg | 4.4 mg |
| 10 | Magnesium Stearate | 3.2 mg | 4.4 mg |
| | Total | 320.0 mg | 440.0 mg |

The immediate release portion of this formulation was formed by mixing items 1 through 6, in the above-listed amounts, in a high shear mixer to obtain a homogeneous mixture: The mixture was then granulated in water and dried in a dryer. The dried granular mass was then milled and then items 9 and 10 were added by blending. The lubricated granular mass was then compressed into mini-tablets using a tablet press for individual tablet weight of 160 mg. The foregoing steps are conventional steps used in the tablet forming industry.

The sustained release portion was formed by mixing items 1 through 3, and 7 and 8, in the amounts listed in the table above, using a laboratory high shear mixer to obtain a homogenous mixture. The mixture was granulated in water and dried in a dryer. The dried granular mass was then milled followed by blending with the item 9 for three minutes followed by blending with item 10 for an additional two minutes in a V-blender. The lubricated granular mass was then compressed into mini-tablets using a tablet press to obtain an individual tablet weight of 220 mg. The foregoing steps are conventional steps used in the tablet forming industry.

The mini-tablets were encapsulated in a capsule containing two immediate release mini-tablets and two sustained release mini-tablets.

### EXAMPLE 2

### Preparation of Sustained Released Dosage Forms Containing Acetaminophen and Tramadol

TABLE 2
Composition (%) of single unit immediate-release and sustained-release
tablets, respectively, containing Acetaminophen and Tramadol

| | | Immediate | | Sustained | |
|---|---|---|---|---|---|
| Ingredient | | Release | | Release | |
| 1 | Acetaminophen | 250.0 | mg | 250.0 | mg |
| 2 | Tramadol Hydrochloride | 25.0 | mg | 25.0 | mg |
| 3 | Microcrystalline Cellulose | 36.4 | mg | 37.8 | mg |
| 4 | Povidone K-90 | 18.0 | mg | 13.8 | mg |
| 5 | Starch | 18.0 | mg | — | |
| 6 | Croscarmellose Sodium | 5.4 | mg | — | |
| 7 | Sodium Alginate (Keltone LV) | — | | 36.8 | mg |
| 8 | Hydroxypropyl Methylcellulose | — | | 41.4 | mg |
| | (Methocel K4M) | | | | |
| 9 | Colloidal Silicon Dioxide | 3.6 | mg | 4.6 | mg |
| 10 | Magnesium Stearate | 3.6 | mg | 4.6 | mg |
| 11 | Copolymer of Methacrylic Acid | — | | 30.0 | mg |
| | (Eudragit L30D) | | | | |
| 12 | Talc | — | | 11.4 | mg |
| 13 | Triethyl Citrate | — | | 4.6 | mg |
| 14 | Purified Water | — | | (80 | mg) |
| | Total | 360.0 | mg | 460.0 | mg |

The immediate release portion of the formulation was formed by mixing items 1 through 6, in the amounts listed in the above formulation, in a high shear mixer to obtain a homogenous mixture. The mixture was then granulated in water or other suitable granulation fluids and dried in a tray dryer at 60° C. for about one hour. The dried granular mass was milled followed by blending with item 9 for three minutes followed by blending with the item 10 for an additional two minutes in a V-blender. The lubricated granular mass was then compressed into mini-tablets using a tablet press for individual tablet weights of 180 mg. The foregoing steps are conventional steps used in the tablet forming industry.

The sustained release portion of the formulation was formed by mixing 1 through 4, 7, and 8 in the amounts listed in the above formulation, using a laboratory high shear mixer to obtain a homogenous mixture. The mixture was then granulated in water and dried in a tray dryer at 60° C. for about one hour. The dried granular mass was then milled followed by blending with item 9 for three minutes followed by blending with the item 10 for an additional two minutes in a V-blender. After blending, the lubricated granular mass was compressed into mini-tablets using a tablet press to obtain individual tablet weights of 207 mg. The tablets were coated with items 11 through 14 to target a weight gain of 13 mg. The foregoing steps are conventional steps used in the tablet forming industry.

The mini-tablets were encapsulated in a capsule containing 2 immediate release mini-tablets and 2 sustained release mini-tablets.

### EXAMPLES 3 AND 4

### Preparation of Sustained Released Dosage Forms Containing Acetaminophen and Tramadol

TABLE 3
Composition (%) of single unit sustained-release tablets
containing Acetaminophen and Tramadol

| Ingredient | | Example 3 | | Example 4 | |
|---|---|---|---|---|---|
| 1 | Acetaminophen (Micronized) | 325.0 | mg | 390.0 | mg |
| 2 | Tramadol Hydrochloride | 37.5 | mg | 45.0 | mg |
| 3 | Microcrystalline Cellulose | 22.5 | mg | 44.0 | mg |
| 4 | Povidone K-30 | 20.0 | mg | 24.0 | mg |
| 5 | Hydroxypropyl Methylcellulose | 40.0 | mg | 40.0 | mg |
| | (Methocel K100LV) | | | | |
| 6 | Hydroxypropyl Methylcellulose | 45.0 | mg | 45.0 | mg |
| | (Methocel K4M) | | | | |
| 7 | Colloidal Silicon Dioxide | 5.0 | mg | 6.0 | mg |
| 8 | Magnesium Stearate | 5.0 | mg | 6.0 | mg |
| 9 | Copolymer of Methacrylic Acid | 32.5 | mg | — | |
| | (Eudragit L30D) | | | | |
| 10 | Talc | 12.2 | mg | — | |
| 11 | Triethyl Citrate | 5.3 | mg | — | |
| 12 | Purified Water | (81 | mg) | | |
| 13 | Acetaminnophen (Micronized) | 325.0 | mg | 260 | mg |
| 14 | Tramadol Hydrochloride | 37.5 | mg | 30.0 | mg |
| 15 | Hydroxypropyl Methylcellulose | 17.5 | mg | 10.0 | mg |
| | (Methocel E5) | | | | |
| | Total | 930.0 | mg | 900.0 | mg |

### EXAMPLE 3

Items 1 through 6 listed in the above formulation were mixed in the specified quantities using a high shear mixer to obtain a homogenous mixture. The mixture was then granulated in water and dried in a tray dryer at 60° C. for about one hour. The dried granular mass was then milled followed by blending with item 7 for three minutes followed by blending with item 8 for an additional two minutes in a V-blender. The lubricated granular mass was compressed into tablets using a tablet press to obtain an individual tablet weight of 500 mg. Following the compression, the tablets were coated with the items 9 through 12 to achieve a target weight gain of approximately 50 mg or 10% weight gain. After completion of the enteric coating, the product was further coated with an active drug layer using a suspension formulation containing the items 13 through 15. The finished product may be further clear or color coated using a conventional film coating process.

### EXAMPLE 4

The lubricated granular mass formed in Example 3 was then compressed into tablets using a tablet press to obtain an individual tablet weight of 600 mg. The tablets were coated with the items 13 through 15 in a suspension to form an immediate release drug layer. The finished product may be further clear or color coated using a conventional film coating process. The dissolution testing was performed using USP apparatus II (Paddle Method) at 50 rpm for the first hour in a simulated gastric fluid and for the second hour and after in a simulated intestinal fluid. The drug release from the preferred formulations above is as follows:

TABLE 4
Percent Drug Released from Dosage Forms

| | Example 1 | | Example 2 | |
|---|---|---|---|---|
| Time | Acetaminophen | Tramadol | Acetaminophen | Tramadol |
| 0 | 0 | 0 | 0 | 0 |
| 1 | 52.8 ± 3.9 | 55.0 ± 4.8 | 49.8 ± 5.6 | 50.8 ± 5.2 |
| 2 | 64.2 ± 4.8 | 67.1 ± 4.3 | 56.7 ± 6.0 | 61.9 ± 4.8 |
| 4 | 85.1 ± 4.0 | 88.4 ± 3.7 | 82.9 ± 4.8 | 83.6 ± 4.3 |
| 6 | 98.0 ± 3.1 | 97.9 ± 2.9 | 96.7 ± 3.9 | 96.2 ± 3.4 |
| 8 | 100.6 ± 2.2 | 100.0 ± 2.4 | 102.4 ± 2.5 | 99.8 ± 2.8 |

TABLE 5
Percent Drug Released from Dosage Forms

| | Example 3 | | Example 4 | |
|---|---|---|---|---|
| Time | Acetaminophen | Tramadol | Acetaminophen | Tramadol |
| 0 | 0 | 0 | 0 | 0 |
| 1 | 49.6 ± 6.2 | 49.8 ± 5.5 | 53.1 ± 5.3 | 55.5 ± 4.5 |
| 2 | 65.5 ± 5.1 | 66.4 ± 4.6 | 68.2 ± 4.8 | 71.4 ± 4.2 |
| 4 | 82.8 ± 4.8 | 84.8 ± 3.7 | 89.0 ± 4.2 | 89.6 ± 4.0 |
| 6 | 97.1 ± 3.5 | 97.7 ± 2.5 | 98.1 ± 3.6 | 99.0 ± 3.1 |
| 8 | 101.3 ± 2.6 | 101.0 ± 2.3 | 100.7 ± 2.7 | 101.3 ± 2.6 |

### EXAMPLE 5

### Preparation of Sustained Released Dosage Forms Containing Acetaminophen and Tramadol

TABLE 6
Composition (%) of single unit immediate-release and
sustained-release tablets, respectively, containing
Acetaminophen and Tramadol

| | | Immediate | | Sustained | |
|---|---|---|---|---|---|
| Ingredient | | Release | | Release | |
| 1 | Acetaminophen | 273.0 | mg | 377.0 | mg |
| 2 | Tramadol Hydrochloride | 16.5 | mg | 58.5 | mg |
| 3 | Microcrystalline Cellulose | 20.95 | mg | 45.5 | mg |
| 4 | Hydroxypropyl Methylcellulose | — | mg | 110.0 | mg |
| | (Methocel K4M) | | | | |
| 5 | Hydroxypropylcellulose (HPC) EXF | 13.05 | mg | 23.0 | mg |
| | (Klucel EXF) | | | | |
| 6 | Cross-linked Carboxymethylcellulose | 5.5 | mg | — | mg |
| | Sodium (CMC-Na) | | | | |
| 7 | Hydroxypropyl Methylcellulose E-6 | 6.0 | mg | — | mg |
| | (HPMC E-6) | | | | |
| 8 | Magnesium Stearate | 4.0 | mg | 8.0 | mg |
| | Total | 339.0 | mg | 622.0 | mg |

The immediate release portion of the formulation was formed by mixing items 1-3, a portion of item 6, and item 7, in the amounts listed in the above formulation, in a high shear mixer to obtain a homogenous mixture. The mixture was then granulated in water or other suitable granulation fluids and the wet granules were passed through a mesh screen and dried in a tray dryer at 60° C. for about one hour. The dried granular mass was then milled and passed through a mesh screen before blending. The granules were blended with item 5 and the remainder of item 6 for about five minutes followed by blending with item 8 for an additional two minutes in a V-blender. The lubricated granules were later used in a compression.

The sustained release portion of the formulation was formed by mixing items 1 through 4, in the amounts listed in the above formulation, using a laboratory high shear mixer to obtain a homogenous mixture. The mixture was then granulated in water and the wet granules were passed through a mesh screen and dried in a tray dryer at 60° C. for about one hour. The dried granular mass was then milled and passed through a mesh screen prior to blending. The granules were then blended with item 5 for about five minutes followed by blending with item 8 for additional two minutes in a V-blender. The lubricated granules were later used for compression.

After the granulation process, the immediate-release granules and the sustained-release granules were compressed into a bilayer tablet with total tablet weight of 961 mg, of which 339 mg for immediate-release layer and 622 mg for sustained-release layer, using a multilayer rotary press. The foregoing steps are conventional steps used in the tablet manufacturing industry.

The dissolution tests on the resulting bilayer tablets were performed using the USP dissolution method II at the paddle speed of 75 rpm and the simulated intestinal fluid as the dissolution medium. The dissolution test results are summarized in Table 9.

### EXAMPLE 6

### Preparation of Sustained Released Dosage Forms Containing Acetaminophen and Tramadol

TABLE 7
Composition (%) of single unit immediate-release and
sustained-release tablets, respectively, containing
Acetaminophen and Tramadol

| | | Immediate | | Sustained | |
|---|---|---|---|---|---|
| Ingredient | | Release | | Release | |
| 1 | Acetaminophen | 227.5 | mg | 422.5 | mg |
| 2 | Tramadol Hydrochloride | 12.0 | mg | 63.0 | mg |
| 3 | Microcrystalline Cellulose | 13.7 | mg | 63.2 | mg |
| 4 | Hydroxypropyl Methylcellulose (Methocel | — | mg | 36.3 | mg |
| | K100lv) | | | | |
| 5 | Hydroxypropyl Methylcellulose | — | mg | 46.0 | mg |
| | (Methocel K4M) | | | | |
| 6 | Hydroxypropylcellulose (HPC) EXF | 10.0 | mg | 21.0 | mg |
| | (Klucel EXF) | | | | |
| 7 | Cross-linked Carboxymethylcellulose | 5.8 | mg | — | mg |
| | Sodium (CMC-Na) | | | | |
| 8 | Hydroxypropyl Methylcellulose E-6 | 6.0 | mg | — | mg |
| | (HPMC E-6) | | | | |
| 9 | Magnesium Stearate | 3.0 | mg | 8.0 | mg |
| | Total | 278.0 | mg | 660.0 | mg |

The immediate release portion of the formulation was formed by mixing items 1-3, a portion of item 7, and item 8, in the amounts listed in the above formulation, in a high shear mixer to obtain a homogenous mixture. The mixture was then granulated in water or other suitable granulation fluids and the wet granules were passed through a mesh screen and dried in a tray dryer at 60° C. for about one hour. The dried granular mass was then milled and passed through a mesh screen prior to blending. The granules were blended with item 6 and the remainder of item 7 for about five minutes followed by blending with item 9 for an additional two minutes in a V-blender. The lubricated granules were later used in a compression.

The sustained release portion of the formulation was formed by mixing items 1 through 5, in the amounts listed in the above formulation, using a high shear mixer to obtain a homogenous mixture. The mixture was then granulated in water and the wet granules were passed through a mesh screen and dried in a tray dryer at 60° C. for about one hour. The dried granular mass was then milled and passed through a mesh screen prior to blending. The granules were then blended with item 6 for about five minutes followed by blending with item 9 for an additional two minutes in a V-blender. The lubricated granules were later used for compression.

After the granulation process, the immediate-release granules and the sustained-release granules were compressed into a bilayer tablet with total tablet weight of 938 mg, of which 278 mg for immediate-release layer and 660 mg for sustained-release layer using a multilayer rotary press. The foregoing steps are conventional steps used in the tablet manufacturing industry.

The dissolution tests on the resulting bilayer tablets were performed using the USP dissolution method II at the paddle speed of 75 rpm and the simulated intestinal fluid as the dissolution medium. The dissolution test results are summarized in Table 9.

### EXAMPLE 7

### Preparation of Sustained Released Dosage Forms Containing Acetaminophen and Tramadol

TABLE 8
Composition (%) of single unit immediate-release and
sustained-release tablets, respectively, containing
Acetaminophen and Tramadol

| | | Immediate | | Sustained | |
|---|---|---|---|---|---|
| Ingredient | | Release | | Release | |
| 1 | Acetaminophen | 195.0 | mg | 455.0 | mg |
| 2 | Tramadol Hydrochloride | 18.75 | mg | 56.25 | mg |
| 3 | Microcrystalline Cellulose | 10.0 | mg | — | mg |
| 4 | Hydroxypropyl Methylcellulose | — | mg | 115.0 | mg |
| | (Methocel K4M) | | | | |
| 5 | Eudragit L100-55 | — | mg | 35.5 | mg |
| 6 | Cross-linked Carboxymethylcellulose | 5.0 | mg | — | mg |
| | Sodium (CMC-Na) | | | | |
| 7 | Hydroxypropyl Methylcellulose E-6 | 5.5 | mg | — | mg |
| | (HPMC E-6) | | | | |
| 8 | Magnesium Stearate | 2.0 | mg | 12.0 | mg |
| | Total | 236.25 | mg | 673.75 | mg |

The immediate release portion of the formulation was formed by mixing items 1-3, 6 and 7, in the amounts listed in the above formulation, in a high shear mixer to obtain a homogenous mixture. The mixture was then granulated in water or other suitable granulation fluids and the wet granules were passed through a mesh screen and dried in a tray dryer at 60° C. for about one hour. The dried granular mass was then milled and passed through a mesh screen prior to blending. The granules were blended with item 8 for two minutes in a V-blender. The lubricated granules were later used in a compression.

The sustained release portion of the formulation was formed by mixing items 1, 2, and 4, in the amounts listed in the above formulation, using a high shear mixer to obtain a homogenous mixture. The mixture was then granulated with a pre-dissolved granulation solvent of 65%/35% (w/w) ethanol/water containing item 5. The wet granules were passed through a mesh screen and dried in a tray dryer at 60° C. for about one hour. The dried granular mass was then milled and passed through a mesh screen prior to blending. The granules were then blended with item 8 for two minutes in a V-blender. The lubricated granules were later used for compression.

After the granulation process, the immediate-release granules and the sustained-release granules were compressed into a bilayer tablet with total tablet weight of 910 mg of which 236.25 mg for immediate-release layer and 673.75 mg for sustained-release layer, using a multilayer rotary press. The foregoing steps are conventional steps used in the tablet manufacturing industry.

The dissolution tests on the resulting bilayer tablets were performed using the USP dissolution method II at the paddle speed of 75 rpm and the simulated intestinal fluid as the dissolution medium. The dissolution test results are summarized in Table 10.

TABLE 9
Percent Drug Released from Dosage Forms

| | Example 5 | | Example 6 | |
|---|---|---|---|---|
| Time | Acetaminophen | Tramadol | Acetaminophen | Tramadol |
| 0 | 0 | 0 | 0 | 0 |
| 1 | 48.1 ± 1.7 | 40.9 ± 1.5 | 42.3 ± 1.4 | 37.2 ± 0.6 |
| 2 | 54.4 ± 1.0 | 51.2 ± 1.0 | 50.2 ± 1.0 | 48.9 ± 1.0 |
| 4 | 61.7 ± 1.3 | 64.6 ± 1.4 | 60.6 ± 1.6 | 64.7 ± 1.8 |
| 6 | 67.5 ± 1.7 | 74.2 ± 1.9 | 71.1 ± 0.4 | 78.0 ± 0.9 |
| 8 | 72.2 ± 2.1 | 81.5 ± 2.3 | 78.4 ± 0.6 | 86.8 ± 1.3 |
| 10 | 76.7 ± 2.3 | 87.2 ± 2.7 | 85.0 ± 0.4 | 91.5 ± 0.5 |
| 12 | 80.3 ± 2.8 | 91.2 ± 3.2 | 91.1 ± 0.6 | 94.8 ± 0.6 |

TABLE 10
Percent Drug Released from Dosage Forms

Example 7

| Time | Acetaminophen | Tramadol |
|---|---|---|
| 0 | 0 | 0 |
| 1 | 39.3 ± 2.4 | 38.2 ± 0.9 |
| 2 | 45.4 ± 1.0 | 46.3 ± 1.0 |
| 4 | 55.8 ± 1.6 | 58.2 ± 1.5 |
| 6 | 63.8 ± 1.8 | 67.2 ± 1.7 |
| 8 | 70.9 ± 2.1 | 74.6 ± 2.0 |
| 10 | 77.2 ± 2.4 | 81.1 ± 2.2 |
| 12 | 82.8 ± 2.6 | 86.2 ± 2.1 |

### EXAMPLE 8

### In Vivo Bioavailability Study in Humans

The in vivo bioavailability study in humans of the dosage forms prepared according to Examples 5 and 6 was conducted using a commercial immediate release dosage form as a reference (Ultracet® Tablets). The study was an open-label, randomized three way crossover design. Night healthy volunteers were enrolled and eight subjects completed the study. The drug administration was separated by two washout periods of at least seven (7) days each. Following an overnight fasting, each treatment group received each of the three following treatments during the course of the study.
Regimen A: A sustained/modified release dosage form, Formulation T1 (Example 5), one tablet containing 75 mg Tramadol and 650 mg Acetaminophen, having a T₉₀ value of approximately 12 hours
Regimen B: A sustained/modified release dosage form, Formulation T2 (Example 6), one tablet containing 75 mg Tramadol and 650 mg Acetaminophen, having a T₉₀ value of approximately 10 hours
Regimen C: The reference formulation, Ultracet®, an immediate release product containing 37.5 mg Tramadol and 325 mg Acetaminophen, administered twice with a 6 hours interval.

Blood samples were collected from each subject receiving treatment regimens A and B for pharmacokinetic sampling at approximate times after administration as follows: 0, 0.25, 0.5, 0.75, 1, 1.5, 1.75, 2, 2.5, 3, 4, 5, 6, 8, 10, 12, 16, 24, 36 and 48 hours. For subjects receiving treatment regimen C, blood samples were collected at approximate times after administration of the first dose as follows: 0, 0.25, 0.5, 0.75, 1, 1.5, 1.75, 2, 2.5, 3, 4, 5, 6, 6.25, 6.5, 6.75, 7, 7.5, 8, 9, 10, 11, 12, 16, 24, 36 and 48 hours.

Plasma concentrations of acetaminophen, tramadol and M1, the active metabolite of Tramadol were determined using a validated LC/MS/MS method. Values for the pharmacokinetic (PK) parameters of acetaminophen, tramadol and M1 were estimated using noncompartmental methods. All the PK parameters reported in the Table 11 were calculated using a computing software. The plasma concentration-time profiles of acetaminophen, Tramadol and M1 for all three treatment regimens are shown in FIGS. 1 through 3, respectively.

As shown in FIGS. 1-3 and Table 11, volunteers received one tablet of each of two test formulations exhibited a rapid rise in plasma concentrations of acetaminophen (FIG. 1), tramadol (FIG. 2) and its active metabolite (FIG. 3), M1, which were similar to or higher than those of the reference formulation within about 1.5 hour after oral administration to provide a fast on-set analgesic effect. Subsequent to the fast onset plasma levels due to the release of acetaminophen and tramadol from the immediate release layer, the longer Tmax and prolonged plasma concentrations of acetaminophen, tramadol and its active metabolite, M1 were achieved and maintained at the levels similar to or higher than those of the reference formulation for at least 8 to 12 hours, thus providing therapeutic efficacy (Tables 12 and 13). Both test formulations showed the total amount of exposure of acetaminophen, tramadol and M1 similar to those of the reference formulation given twice in every 6 hours. The important pharmacokinetic parameters of two test formulations and the reference formulation are summarized in Tables 11 through 14.

TABLE 11
Summary of Plasma Concentrations of Acetaminophen (APAP),
Tramadol (TMD) and M1 at the Tₘₐₓ of the first IR
(Reference) Formulation Dose Following Single Dose Oral
Administration of Sustained-Release Formulations (T1 and T2),
and Immediate-Release Formulation (Reference IR) in Healthy
Volunteers (Mean ± SD, n = 8)

| | Cₘₐₓ* (ng/mL) | Cₘₐₓ* (ng/mL) | Cₘₐₓ* (ng/mL) |
|---|---|---|---|
| | Regimen A | Regimen A | Regimen C |
| | T1 | T2 | IR Reference |
| APAP | 2553.1 ± 968.6 | 2605.5 ± 964.8 | 2809.8 ± 467.5 |
| TMD | 57.6 ± 13.4 | 78.0 ± 23.7 | 76.6 ± 15.5 |
| M1 | 20.2 ± 6.8 | 20.6 ± 7.1 | 19.0 ± 5.9 |

TABLE 12
Summary of Tₘₐₓ Values of Acetaminophen (APAP),
Tramadol (TMD) and M1 Following Single Dose Oral
Administration of Sustained-Release Formulations (T1 and T2),
and Immediate-Release Formulation (Reference IR) in Healthy
Volunteers (Mean ± SD, n = 8)

| | Tₘₐₓ (hr) | Tₘₐₓ (hr) | Tₘₐₓ (hr) |
|---|---|---|---|
| | Regimen A | Regimen A | Regimen C |
| | T1 | T2 | IR Reference |
| APAP | 1.59 ± 0.73 | 1.84 ± 1.13 | 0.97 ± 0.53 |
| TMD | 5.25 ± 1.28 | 5.38 ± 0.74 | 1.38 ± 0.73 |
| M1 | 5.50 ± 1.07 | 6.38 ± 2.33 | 2.47 ± 1.70 |

TABLE 13
Summary of Plasma Concentrations of Acetaminophen (APAP),
Tramadol (TMD) and M1 at 12 hours and 6 hours Following Single
Dose Oral Administration of Sustained-Release Formulations
(T1 and T2), and Immediate-Release Formulation (Reference IR),
respectively, in Healthy Volunteers (Mean ± SD, n = 8)

| | | | C_{6 h} |
|---|---|---|---|
| | C_{12 h} | C_{12 h} | (ng/mL) |
| | (ng/mL) | (ng/mL) | Regimen C, IR |
| | Regimen A, T1 | Regimen A, T2 | Reference |
| APAP | 584.0 ± 236.3 | 549.0 ± 142.7 | 784.4 ± 223.4 |
| TMD | 57.8 ± 20.3 | 67.4 ± 17.8 | 49.7 ± 10.4 |
| M1 | 18.3 ± 5.1 | 21.4 ± 8.0 | 15.1 ± 4.8 |

TABLE 14
Summary of Plasma Exposures of Acetaminophen (APAP),
Tramadol (TMD) and M1Following Single Dose Oral
Administration of Formulations T1 and T2, and Reference
Formulation in Healthy Volunteers (Mean ± SD, n = 8)

| | | | AUC_{0~∞} |
|---|---|---|---|
| | AUC_{0~∞} | AUC_{0~∞} | (ng * h/mL) |
| | (ng * h/mL) | (ng * h/mL) | Regimen C, |
| | Regimen A, T1 | Regimen B, T2 | IR Reference |
| APAP | 27764 ± 7436 | 27705 ± 4223 | 29492 ± 5178 |
| F (%)* | 94 ± 15 | 95 ± 14 | |
| TMD | 1761 ± 681 | 1892 ± 435 | 1766 ± 626 |
| F (%)* | 101 ± 15 | 112 ± 19 | |
| M1 | 518 ± 120 | 531 ± 168 | 484 ± 129 |
| F (%)* | 109 ± 16 | 109 ± 21 | |

*Relative Bioavailability of the test formulation as compared to the reference formulation

### EXAMPLE 9

### Prophetic Example for Preparation of Sustained Released Dosage Forms Containing Acetaminophen and Tramadol, A Once-a-Day Formulation

TABLE 15
Composition (%) of single unit immediate-release and
sustained-release tablets, respectively, containing
Acetaminophen and Tramadol

| | | Immediate | | Sustained | |
|---|---|---|---|---|---|
| Ingredient | | Release | | Release | |
| 1 | Acetaminophen | 273.0 | mg | 377.0 | mg |
| 2 | Tramadol Hydrochloride | 13.5 | mg | 61.5 | mg |
| 3 | Microcrystalline Cellulose | 12.0 | mg | 24.5 | mg |
| 4 | Hydroxypropyl Methylcellulose (Methocel | — | mg | 60.0 | mg |
| | K100lv) | | | | |
| 5 | Hydroxypropyl Methylcellulose | — | mg | 200.0 | mg |
| | (Methocel K4M) | | | | |
| 6 | Hydroxypropylcellulose (HPC) EXF | 10.0 | mg | 23.0 | mg |
| | (Klucel EXF) | | | | |
| 7 | Cross-linked Carboxymethylcellulose | 5.5 | mg | — | mg |
| | Sodium (CMC-Na) | | | | |
| 8 | Hydroxypropyl Methylcellulose E-6 | 6.0 | mg | — | mg |
| | (HPMC E-6) | | | | |
| 9 | Magnesium Stearate | 4.0 | mg | 8.0 | mg |
| | Total | 324.0 | mg | 754.0 | mg |

The immediate release portion of the formulation would be formed by mixing items 1-3, a portion of item 7, and item 8, in the amounts listed in the above formulation, in a high shear mixer to obtain a homogenous mixture. The mixture would then granulated in water or other suitable granulation fluids and the wet granules would be passed through a mesh screen and dried in a tray dryer at 60° C. for about one hour. The dried granular mass would then milled and passed through a mesh screen prior to blending. The granules would then be blended with item 6 and the remainder of item 7 for about five minutes followed by blending with item 9 for an additional two minutes in a V-blender. The lubricated granules would be used in a compression.

The sustained release portion of the formulation would be formed by mixing items 1 through 5, in the amounts listed in the above formulation, using a high shear mixer to obtain a homogenous mixture. The mixture would then granulated in water and the wet granules would be passed through a mesh screen and dried in a tray dryer at 60° C. for about one hour. The dried granular mass would then be milled and passed through a mesh screen prior to blending. The granules would then be blended with item 6 for about five minutes followed by blending with item 9 for an additional two minutes in a V-blender. The lubricated granules would be used in a compression run.

After the granulation process, the immediate-release granules and the sustained-release granules would be compressed into a bilayer tablet with total tablet weight of 1078 mg, of which 324 mg would be for immediate-release layer and 754 mg would be for sustained-release layer using a multilayer rotary press. The foregoing steps are conventional steps used in the tablet manufacturing industry.

The dosage form produced by this manufacture is designed to deliver 650 mg of acetaminophen and 75 mg of tramadol per unit for a 24 hour delivery and the patient will take two units to achieve sufficient therapeutic efficacy for approximately 16-24 hours.

### EXAMPLE 10

### Prophetic Example for Preparation of Sustained Released Dosage Forms Containing Acetaminophen and Tramadol

TABLE 16
Composition (%) of single unit immediate-release and
sustained-release tablets, respectively, containing
Acetaminophen and Tramadol

| | | Immediate | | Sustained | |
|---|---|---|---|---|---|
| Ingredient | | Release | | Release | |
| 1 | Acetaminophen | 195.0 | mg | 455.0 | mg |
| 2 | Tramadol Hydrochloride | 22.5 | mg | 52.5 | mg |
| 3 | Microcrystalline Cellulose | 22.6 | mg | 14.5 | mg |
| 4 | Hydroxypropyl Methylcellulose (Methocel | — | mg | 180.0 | mg |
| | K4M) | | | | |
| 5 | Eudragit L100-55 | — | mg | 60.0 | mg |
| 6 | Cross-linked Carboxymethylcellulose | 5.6 | mg | — | mg |
| | Sodium (CMC-Na) | | | | |
| 7 | Hydroxypropyl Methylcellulose E-6 | 6.0 | mg | — | mg |
| | (HPMC E-6) | | | | |
| 8 | Hydroxypropylcellulose (HPC) EXF | 10.0 | mg | 23.0 | mg |
| | (Klucel EXF) | | | | |
| 9 | Magnesium Stearate | 3.3 | mg | 8.0 | mg |
| | Total | 265.00 | mg | 793.0 | mg |

The immediate release portion of the formulation would be formed by mixing items 1-3, 6 and 7, in the amounts listed in the above formulation, in a high shear mixer to obtain a homogenous mixture. The mixture would then granulated in water or other suitable granulation fluids and the wet granules would be passed through a mesh screen and dried in a tray dryer at 60° C. for about one hour. The dried granular mass would then be milled and passed through a mesh screen prior to blending. The granules would then be blended with item 8 for about five minutes followed by blending with item 9 for an additional two minutes in a V-blender. The lubricated granules would be used in a compression.

The sustained release portion of the formulation would be formed by mixing items 1 through 4, in the amounts listed in the above formulation, using a high shear mixer to obtain a homogenous mixture. The mixture would then be granulated with a pre-dissolved granulation solvent of 65%/35% (w/w) ethanol/water containing item 5. The wet granules would then be passed through a mesh screen and dried in a tray dryer at 60° C. for about one hour. The dried granular mass would then be milled and passed through a mesh screen prior to blending. The granules would be blended with item 8 for about five minutes followed by blending with item 9 for an additional two minutes in a V-blender. The lubricated granules would be used in a compression.

After the granulation process, the immediate-release granules and the sustained-release granules would be compressed into a bilayer tablet with total tablet weight of 1058 mg of which 265 mg for immediate-release layer and 793 mg for sustained-release layer, using a multilayer rotary press. The foregoing steps are conventional steps used in the tablet manufacturing industry.

The dosage form that would be produced by this manufacture is designed to deliver 650 mg of acetaminophen and 75 mg of tramadol per unit for a 24 hour delivery and the patient would take two units to achieve sufficient therapeutic efficacy for approximately 16-24 hours.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims

We claim:

1. A pharmaceutical dosage form for sustained release of a combination of acetaminophen of from 100 mg to 1,000 mg and tramadol or its salts of from 15 mg to 150 mg comprising:
a) an immediate release portion comprising about 16%-75% of the total effective amount of the acetaminophen and the tramadol in the form selected from pellets, beads, granules and mini-tablets;
b) a sustained release portion comprising:
a. about 25%-84% of the total effective amount of the acetaminophen and the tramadol in the form selected from pellets, beads, granules and mini-tablets;
b. at least one gelling polymer in an amount by weight of the total formulation of about 6% to 50%;
c) the dosage form releases about 25% to about 60% of the acetaminophen and the tramadol in the first hour, and not less than about 80% of the acetaminophen and the tramadol in the first 24 hours in an intestinal fluid dissolution media using USP dissolution method II with the paddle speed between 50 rpm and 100 rpm.

2. The dosage form of claim 1 releases:
about 25%-60% of the acetaminophen and the tramadol in the first hour, and not less than about 80% of the acetaminophen and the tramadol in the first 12 hours in an intestinal fluid dissolution media using USP dissolution method II with the paddle speed between 50 rpm and 100 rpm.

3. The dosage form of claim 1 wherein the gelling polymer is a homopolymer, copolymer or terpolymer derived from, substituted and unsubstituted, acrylic, methacrylic, methacyrlic acid, methacrylate, ethacrylic acid, or ethacrylate monomers.

4. The dosage form of claim 1 wherein the gelling polymer has a viscosity within the range of from about 60 to about 7,000,000 centipoises, in a 2% by weight solution at 25° C., as measured by a Brookfield LV viscometer.

5. The dosage form of claim 1 wherein the immediate release portion is enteric coated with acrylic acid polymers, methacrylic acid polymers, cellulose acetate phthalate, cellulose phthalate hydroxy propyl methyl ether, polyvinyl acetate phthalate, hydroxypropyl methylcellulose acetate succinate, cellulose acetate trimellitate, or a shellac.

6. The dosage form of claim 1 is a capsule or a tablet.

7. A dosage form for sustained release of a combination of acetaminophen of from 100 mg to 1,000 mg and tramadol or its salt of from 15 mg to 150 mg comprising:
a) a sustained release portion comprising:
1. about 25%-84% of the total effective amount of the acetaminophen and the tramadol,
2. at least one gelling polymer in an amount by weight of the formulation from about 6% to 50%;
b) an immediate release portion comprising about 16%-75% of the total effective amount of the drugs, layered or compressed on the sustained release portion; and
c) the dosage form releases about 25% to about 60% of the acetaminophen and the tramadol in the first hour, and not less than about 80% of the acetaminophen and the tramadol in the first 24 hours in an intestinal fluid dissolution media using USP dissolution method II with the paddle speed between 50 rpm and 100 rpm.

8. The dosage form of claim 7 releases:
about 25%-60% of the acetaminophen and the tramadol in the first hour, and not less than about 80% of the acetaminophen and the tramadol in the first 12 hours in an intestinal fluid dissolution media using USP dissolution method II with the paddle speed between 50 rpm and 100 rpm.

9. The dosage form of claim 8 wherein the gelling polymer is a homopolymer, copolymer or terpolymer derived from, substituted and unsubstituted, acrylic, methacrylic, methacyrlic acid, methacrylate, ethacrylic acid, or ethacrylate monomers.

10. The dosage form of claim 8 wherein the gelling polymer has a viscosity within the range of from about 60 to about 7,000,000 centipoises, in a 2% by weight solution at 25° C., as measured by a Brookfield LV viscometer.

11. A sustained release dosage form of tramadol and acetaminophen, wherein when administered to a human patient, the dosage form produces a plasma profiles in the human patient comprising:
a fast onset plasma level achieved within less than about two hours; followed by a prolonged plasma level of tramadol and acetaminophen for at least 8-12 hours or 16-24 hours maintaining therapeutic efficacy;
a Cmax for tramadol of between about 0.4 ng/mL/mg to about 4.0 ng/mL/mg, and an AUC for tramadol of between about 8 ng*hr/mL/mg to about 50 ng*hr/mL/mg;
a Cmax of an active metabolite of tramadol of between about 0.15 ng/mL/mg to about 0.9 ng/mL/mg, and an AUC for the active metabolite of tramadol of between about 2.5 ng*hr/mL/mg to about 12 ng*hr/mL/mg; and
a Cmax for acetaminophen of between about 2.0 ng/mL/mg to about 8.0 ng/mL/mg and an AUC for acetaminophen of between about 20 ng*hr/mL/mg to about 80 ng*hr/mL/mg after a single dose.
